(19) United States
(12) Patent Application Publication
(10) Pub. No.: US 2010/0318043 A1
(43) Pub. Date: Dec. 16, 2010

(54) Negative Pressure Wound Therapy Systems Capable of Vacuum Measurement Independent of Orientation

(75) Inventors: Arnaz Malhi, Watertown, MA (US); Jesse E. Denson, Lincoln, RI (US)

Correspondence Address: TYCO HEALTHCARE GROUP LP, 15 HAMPSHIRE STREET, MANSFIELD, MA, 02048, US

(73) Assignee: Tyco Healthcare Group LP, Mansfield, MA (US)

(21) Appl. No.: 12/481,934

(22) Filed: Jun. 10, 2009

(51) Int. Cl. A61M 1/00

(52) U.S. Cl. 604/313

(57) ABSTRACT

A portable negative pressure wound therapy system includes a dressing assembly for positioning over a wound to apply negative pressure to the wound and a canister assembly. The canister assembly includes a control unit, a vacuum source disposed in the control unit, a pressure sensor in communication with a processor unit of the control unit, and a collection canister. The collection canister includes an inlet conduit in fluid communication with the dressing assembly, a first chamber to collect wound fluids, an inlet port coupled to the inlet conduit to introduce the wound fluids from the dressing assembly into the first chamber, a suction port to communicate with the first chamber and the vacuum source, a pressure sensor port to communicate with the first chamber and the pressure sensor. The pressure sensor port is in fluid communication with a “T”-off point between the inlet conduit and the inlet port.

### BACKGROUND

1. Technical Field

The present disclosure relates generally to treating a wound by applying negative pressure to the wound and, more particularly, to negative pressure wound therapy systems capable of measuring vacuum levels independent of the orientation of components.

2. Discussion of Related Art

Negative pressure therapy, also known as suction or vacuum therapy, has been used in treating and healing wounds. Treating an open wound by applying negative pressure, e.g., reduced or sub-atmospheric pressure, to a localized reservoir over a wound has been found to assist in closing the wound by increasing blood circulation at the wound area, stimulating the formation of granulation tissue and promoting the migration of healthy tissue over the wound. Negative pressure therapy may also inhibit bacterial growth by drawing wound fluids from the wound such as exudate, which may tend to harbor bacteria. This technique has proven effective for treating a variety of wound conditions, including chronic or healing-resistant wounds and ulcers, and is also used for other purposes such as post-operative wound care.

Generally, negative pressure therapy provides for a wound covering to be positioned over the wound to facilitate suction at the wound area. A conduit is introduced through the wound covering to provide fluid communication to an external vacuum source, such as a hospital vacuum system or a portable vacuum pump. Atmospheric gas, wound exudate or other fluids may thus be drawn from the reservoir through the fluid conduit to stimulate healing of the wound. Generally, a fluid collection canister for collecting fluids aspirated from the wound is positioned in the suction line between the wound covering and the vacuum source. Exudate drawn from the reservoir through the fluid conduit may thus be deposited into the collection canister, which may be disposable.

During a treatment, vacuum levels within a negative pressure wound therapy (NPWT) system may be monitored and controlled. There are a variety of pressure gages, switches, transducers and transmitters that can be used for measuring vacuum levels. For example, there are mechanical gauges that include a pressure sensing element, e.g., a Bourdon tube or a metallic diaphragm, which flexes elastically under the effect of a pressure difference across the element. There are pressure switches that include mechanical pistons. Some pressure switches use a strain gauge and a diaphragm to detect the strain applied by pressure changes. In general, pressure transducers and transmitters convert the mechanical force of applied pressure into an electric signal output that is linear and proportional to the applied pressure. In a transducer or transmitter, vacuum or pressure changes may cause deflection of an elastic ceramic or metallic diaphragm. This deflection varies electrical characteristics of interconnected circuitry to produce a signal that represents the vacuum level, e.g., in volts (V), millivolt per volt (mV/V) or milliamps (mA).

A NPWT system may not function properly when the entry of exudate into inlets, outlets and conduits between the inlets and outlets degrades the capability to accurately measure vacuum levels within the NPWT system. In portable NPWT devices, which may be worn or carried by a patient, there is a likelihood that the apparatus will shift into various orientations while the patient is ambulating, thereby making the possible entry of exudate into the inlets, outlets and conduits more likely to occur. A need thus exists for a NPWT system that permits the accurate measurement of vacuum levels within the system independent of the orientation of various components of the NPWT system.

### SUMMARY

The present disclosure relates to a portable negative pressure wound therapy apparatus including a dressing assembly for positioning over a wound to apply a negative pressure to the wound and a canister assembly. The canister assembly includes a control unit, a vacuum source disposed in the control unit, a pressure sensor in communication with a processor unit of the control unit, and a collection canister. The collection canister includes an inlet conduit in fluid communication with the dressing assembly, a first chamber to collect wound fluids from the dressing assembly, an inlet port coupled to the inlet conduit to introduce the wound fluids from the dressing assembly into the first chamber, and a suction port to communicate with the first chamber and the vacuum source. The canister assembly also includes a pressure sensor port to communicate with the first chamber and the pressure sensor. The pressure sensor port is in fluid communication with a “T”-off point between the inlet conduit and the inlet port.

### BRIEF DESCRIPTION OF THE DRAWINGS

Objects and features of the presently disclosed negative pressure wound therapy systems will become apparent to those of ordinary skill in the art when descriptions of various embodiments thereof are read with reference to the accompanying drawings, of which:

FIG. 1 is a schematic diagram of an embodiment of a negative pressure wound therapy system in accordance with the present disclosure;

FIG. 2 is a schematic diagram of an embodiment of a negative pressure wound therapy system including a canister assembly in accordance with the present disclosure; and

FIG. 3 is a schematic diagram of the canister assembly of the negative pressure wound therapy system illustrated in FIG. 1 shown with a pressure sensor in fluid communication with a “T”-off point between a canister inlet conduit and a canister inlet port in accordance with the present disclosure.

### DETAILED DESCRIPTION

Hereinafter, embodiments of the presently disclosed negative pressure wound therapy systems will be described with reference to the accompanying drawings. Like reference numerals may refer to similar or identical elements throughout the description of the figures. As used herein, “wound exudate”, or, simply, “exudate”, generally refers to any fluid output from a wound, e.g., blood, serum, and/or pus, etc. As used herein, “fluid” generally refers to a liquid, a gas or both. As used herein, “transmission line” generally refers to any transmission medium that can be used for the propagation of signals from one point to another.

Various embodiments of the present disclosure provide negative pressure wound therapy systems (or apparatus). Embodiments of the presently disclosed negative pressure wound therapy systems are generally suitable for use in applying negative pressure to a wound to facilitate healing of the wound in accordance with various treatment modalities. Embodiments of the presently disclosed negative pressure wound therapy systems are entirely portable and may be worn or carried by the user such that the user may be completely ambulatory during the therapy period. Embodiments of the presently disclosed negative pressure wound therapy apparatus and components thereof may be entirely reusable or may be entirely disposable after a predetermined period of use or may be individually disposable whereby some of the components are reused for a subsequent therapy application.

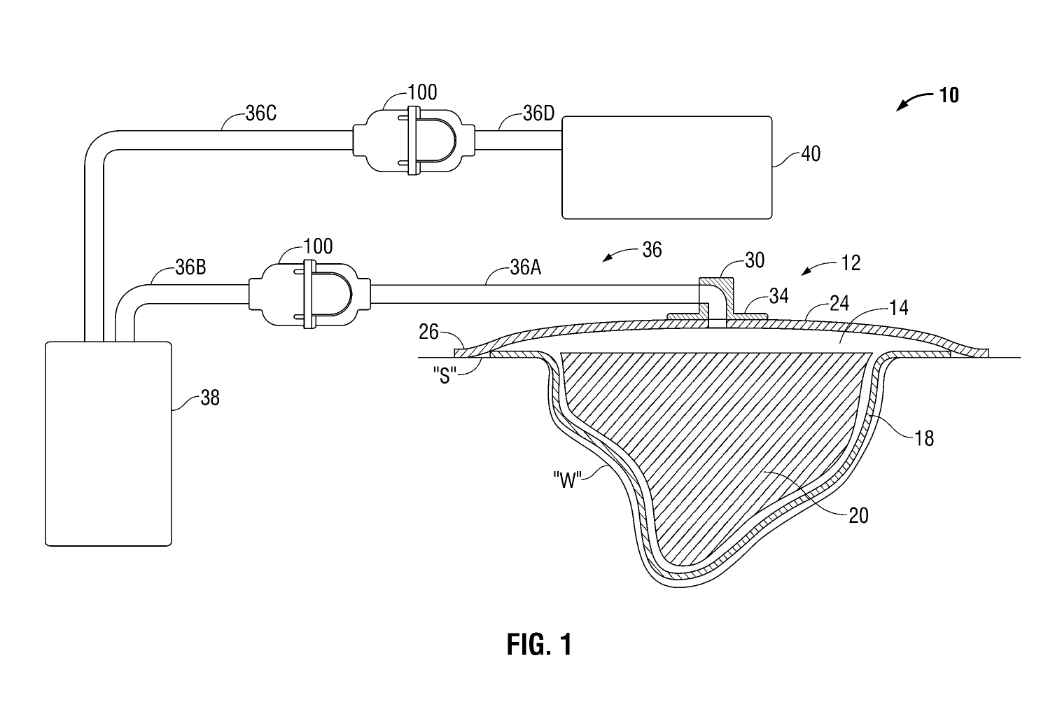

Referring to FIG. 1, a negative pressure wound therapy apparatus according to an embodiment of the present disclosure is depicted generally as 10 for use on a wound bed “w” surrounded by healthy skin “s”. Negative pressure wound therapy apparatus 10 includes a wound dressing 12 positioned relative to the wound bed “w” to define a vacuum chamber 14 about the wound bed “w” to maintain negative pressure at the wound area. Wound dressing 12 includes a contact layer 18, a wound filler 20 and a wound cover 24.

Contact layer 18 is intended for placement within the wound bed “w” and may be relatively non-supportive or flexible to substantially conform to the topography of the wound bed “w”. A variety of materials may be used for the contact layer 18. Contact layer 18 selection may depend on various factors such as the patient's condition, the condition of the periwound skin, the amount of exudate and/or the condition of the wound bed “w”. Contact layer 18 may be formed from perforated film material. The porous characteristic of the contact layer 18 permits exudate to pass from the wound bed “w” through the contact layer 18. Passage of wound exudate through the contact layer 18 may be substantially unidirectional such that exudate does not tend to flow back into the wound bed “w”. Unidirectional flow may be encouraged by directional apertures, e.g., apertures positioned at peaks of undulations or cone-shaped formations protruding from the contact layer 18. Unidirectional flow may also be encouraged by laminating the contact layer 18 with materials having absorption properties differing from those of the contact layer 18, or by selection of materials that promote directional flow. A non-adherent material may be selected for forming the contact layer 18 such that the contact layer 18 does not tend to cling to the wound bed “w” or surrounding tissue when it is removed. One example of a material that may be suitable for use as a contact layer 18 is commercially available under the trademark XEROFLOW® offered by Tyco Healthcare Group LP (d/b/a Covidien). Another example of a material that may be suitable for use as the contact layer 18 is the commercially available CURITY® non-adherent dressing offered by Tyco Healthcare Group LP (d/b/a Covidien).

Wound filler 20 is positioned in the wound bed “w” over the contact layer 18 and is intended to transfer wound exudate. Wound filler 20 is conformable to assume the shape of any wound bed “w” and may be packed up to any level, e.g., up to the level of healthy skin “s” or to overfill the wound such that wound filler 20 protrudes over healthy skin “s”. Wound filler 20 may be treated with agents such as polyhexamethylene biguanide (PHMB) to decrease the incidence of infection and/or other medicaments to promote wound healing. A variety of materials may be used for the wound filler 20. An example of a material that may be suitable for use as the wound filler 20 is the antimicrobial dressing commercially available under the trademark KERLIX™ AMD™ offered by Tyco Healthcare Group LP (d/b/a Covidien).

Cover layer 24 may be formed of a flexible membrane, e.g., a polymeric or elastomeric film, which may include a biocompatible adhesive on at least a portion of the cover layer 24, e.g., at the periphery 26 of the cover layer 24. Alternatively, the cover layer 24 may be a substantially rigid member. Cover layer 24 may be positioned over the wound bed “w” such that a substantially continuous band of a biocompatible adhesive at the periphery 26 of the cover layer 24 forms a substantially fluid-tight seal with the surrounding skin “s”. An example of a material that may be suitable for use as the cover layer 24 is commercially available under the trademark CURAFORM ISLAND® offered by Tyco Healthcare Group LP (d/b/a Covidien). Cover layer 24 may act as both a microbial barrier and a fluid barrier to prevent contaminants from entering the wound bed “w” and to help maintain the integrity thereof.

In one embodiment, the cover layer 24 is formed from a moisture vapor permeable membrane, e.g., to promote the exchange of oxygen and moisture between the wound bed “w” and the atmosphere. An example of a membrane that may provide a suitable moisture vapor transmission rate (MVTR) is a transparent membrane commercially available under the trade name POLYSKIN®II offered by Tyco Healthcare Group LP (d/b/a Covidien). A transparent membrane may help to permit a visual assessment of wound conditions to be made without requiring removal of the cover layer 24.

Wound dressing 12 may include a vacuum port 30 having a flange 34 to facilitate connection of the vacuum chamber 14 to a vacuum system. Vacuum port 30 may be configured as a rigid or flexible, low-profile component and may be adapted to receive a conduit 36 in a releasable and fluid-tight manner. An adhesive on at least a portion of the underside of the flange 34 may be used to provide a mechanism for affixing the vacuum port 30 to the cover layer 24. The relative positions, size and/or shape of the vacuum port 30 and the flange 34 may be varied from an embodiment depicted in FIG. 1. For example, the flange 34 may be positioned within the vacuum chamber 14 such that an adhesive on at least a portion of an upper side surface of the flange 34 affixes the vacuum port 30 to the cover layer 24. A hollow interior portion of the vacuum port 30 provides fluid communication between the conduit 36 and the vacuum chamber 14. Conduit 36 extends from the vacuum port 30 to provide fluid communication between the vacuum chamber 14 and the vacuum source 40. Alternatively, the vacuum port 30 may not be included in the dressing 12 if other provisions are made for providing fluid communication with the conduit 36.

Any suitable conduit may be used for the conduit 36, including conduit fabricated from flexible elastomeric or polymeric materials. In the negative pressure wound therapy apparatus 10 illustrated in FIG. 1, the conduit 36 includes a first conduit section 36A, a second conduit section 36B, a third conduit section 36C and a fourth conduit section 36D. The first conduit section 36A extends from the vacuum port 30 and is coupled via a fluid line coupling 100 to the second conduit section 36B, which extends to the collection canister 38. The third conduit section 36C extends from the collection canister 38 and is coupled via another fluid line coupling 100 to the fourth conduit section 36D, which extends to the vacuum source 40. The shape, size and/or number of conduit sections of the conduit 36 may be varied from the first, second, third and fourth conduit sections 36A, 36B, 36C and 36D depicted in FIG. 1.

The first, second, third and fourth conduit sections 36A, 36B, 36C and 36D of the conduit 36 may be connected to components of the apparatus 10 by conventional air-tight means, such as, for example, friction fit, bayonet coupling, or barbed connectors. The connections may be made permanent. Alternatively, a quick-disconnect or other releasable connection means may be used to provide some adjustment flexibility to the apparatus 10.

Collection canister 38 may be formed of any type of container that is suitable for containing wound fluids. For example, a semi-rigid plastic bottle may be used for the collection canister 38. A flexible polymeric pouch or other hollow container body may be used for the collection canister 38. Collection canister 38 may contain an absorbent material to consolidate or contain the wound fluids or debris. For example, super absorbent polymers (SAP), silica gel, sodium polyacrylate, potassium polyacrylamide or related compounds may be provided within collection canister 38. At least a portion of canister 38 may be transparent or semi-transparent, e.g., to permit a visual assessment of the wound exudate to assist in evaluating the color, quality and/or quantity of exudate. A transparent or semi-transparent portion of the collection canister 38 may permit a visual assessment to assist in determining the remaining capacity or open volume of the canister and/or may assist in determining whether to replace the collection canister 38.

The collection canister 38 is in fluid communication with the wound dressing 12 via the first and second conduit sections 36A, 36B. The third and fourth conduit sections 36C, 36D connect the collection canister 38 to the vacuum source 40 that generates or otherwise provides a negative pressure to the collection canister 38. Vacuum source 40 may include a peristaltic pump, a diaphragmatic pump or other suitable mechanism. Vacuum source 40 may be a miniature pump or micropump that may be biocompatible and adapted to maintain or draw adequate and therapeutic vacuum levels. The vacuum level of subatmospheric pressure achieved may be in the range of about 20 mmHg to about 500 mmHg. In embodiments, the vacuum level may be about 75 mmHg to about 125 mmHg, or about 40 mmHg to about 80 mmHg. One example of a peristaltic pump that may be used as the vacuum source 40 is the commercially available Kangaroo PET Eternal Feeding Pump offered by Tyco Healthcare Group LP (d/b/a Covidien). Vacuum source 40 may be actuated by an actuator (not shown) which may be any means known by those skilled in the art, including, for example, alternating current (AC) motors, direct current (DC) motors, voice coil actuators, solenoids, and the like. The actuator may be incorporated within the vacuum source 40.

In embodiments, the negative pressure wound therapy apparatus 10 may include one or more fluid line couplings 100 that allow for selectable coupling and decoupling of conduit sections. For example, a fluid line coupling 100 may be used to maintain fluid communication between the first and second conduit sections 36A, 36B when engaged, and may interrupt fluid flow between the first and second conduit sections 36A, 36B when disengaged. Thus, fluid line coupling 100 may facilitate the connection, disconnection or maintenance of components of the negative pressure wound therapy apparatus 10, including the replacement of the collection canister 38. Additional or alternate placement of one or more fluid line couplings 100 at any location in line with the conduit 36 may facilitate other procedures. For example, the placement of a fluid line coupling 100 between the third and fourth conduit sections 36C, 36D, as depicted in FIG. 1, may facilitate servicing of the vacuum source 40.

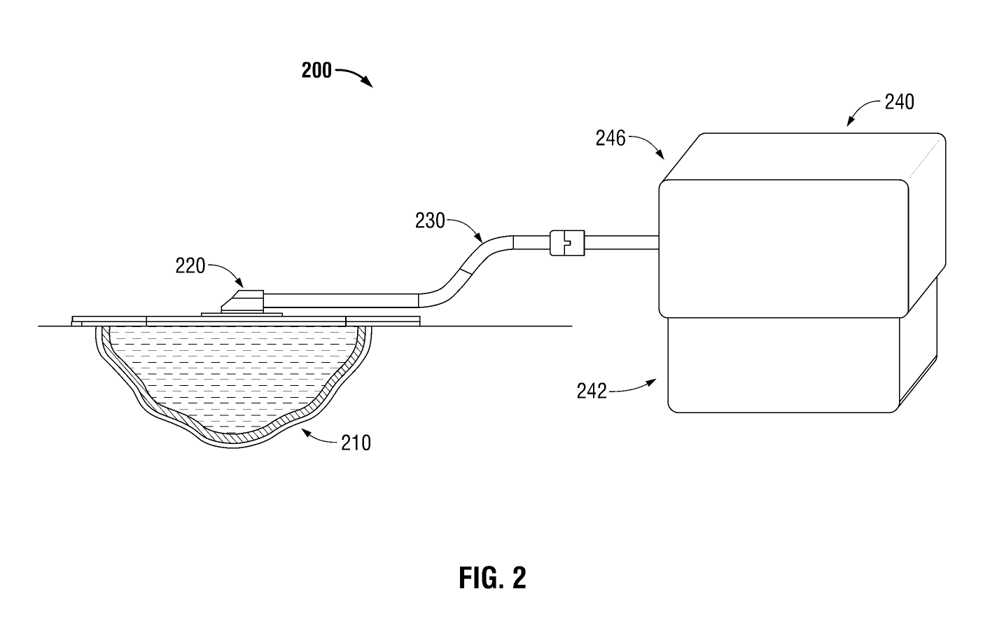

Referring to FIG. 2, the negative pressure wound therapy system shown generally as 200 includes a dressing assembly 210, a wound port assembly 220, an extension assembly 230 and a canister assembly 240. Dressing assembly 210 is positioned relative to the wound area to define a vacuum chamber about the wound area to maintain negative pressure at the wound area. Dressing assembly 210 may be substantially sealed from extraneous air leakage, e.g., using adhesive coverings. Wound port assembly 220 is mounted to the dressing assembly 210. For example, wound port assembly 220 may include a substantially continuous band of adhesive at its periphery for affixing the wound port assembly 220 to the dressing assembly 210. Extension assembly 230 is coupled between the wound port assembly 220 and the canister assembly 240 and defines a fluid flow path between the wound port assembly 220 and the canister assembly 240. A hollow interior of the wound port assembly 220 provides fluid communication between the extension assembly 230 and the interior of the dressing assembly 210. Dressing assembly 210 and the wound port assembly 220 shown in FIG. 2 are similar to components of the wound dressing 12 of FIG. 1 and further description thereof is omitted in the interests of brevity.

Canister assembly 240 includes a control unit 246 and a collection canister 242. In embodiments, the collection canister is disposed below the control unit 246. Control unit 246 and the collection canister 242 may be releasably coupled. Mechanisms for selective coupling and decoupling of the control unit 246 and the collection canister 242 include fasteners, latches, clips, straps, bayonet mounts, magnetic couplings, and other devices. Collection canister 242 may consist of any container suitable for containing wound fluids.

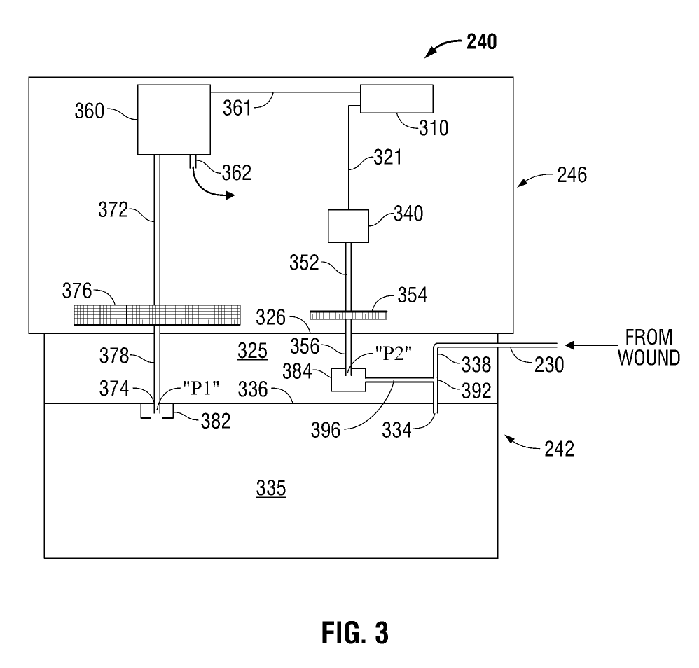

In one embodiment, the negative pressure wound therapy system 200 is capable of operating in a continuous mode or an alternating mode. In the continuous mode, the control unit 246 controls a pump (e.g., 360 shown in FIG. 3) to continuously supply a selected vacuum level at the collection canister 242 to create a reduced pressure state within the dressing assembly 210. In the alternating mode, the control unit 246 controls the pump to alternating supply a first negative pressure, e.g., about 80 mmHg, at the collection canister 242 for a preset fixed amount of time and a second negative pressure, e.g., about 50 mmHg, at the collection canister 242 for a different preset fixed amount of time. In general, the output of the pump is directly related to the degree of air leakage in the negative pressure wound therapy system 200 and the open volume in the collection canister 242. If there is sufficient air leakage in the system 200, e.g., at the dressing assembly 210, the pump can remain on continuously and the control unit 246 can control negative pressure at the collection canister 242 by adjusting the pump speed. Alternatively, if there is not sufficient air leakage in the system 200 to permit the pump to remain on continuously, the control unit 246 can control negative pressure at the collection canister 242 by turning the pump on and off, e.g., for non-equal on/off periods of time.

If an air leak develops in the negative pressure wound therapy system 200, e.g., at the dressing assembly 210, for which the control unit 246 can not compensate by increasing the pump speed, the control unit 246 may indicate an alarm. For example, the control unit 246 may indicate a leak alarm after two consecutive minutes of operation in which the vacuum level is below the current set point (or below the minimum level of a set point range). Audible indicatory means may also be incorporated or associated with the control unit 246 to notify the user of a condition, e.g., leak, canister assembly tip, failed pressure sensor, failed pump, excessive vacuum, low battery conditions, occlusion or system error conditions. The audio indication for some alarm types can be paused by pressing a pause alarm button (not shown).

In embodiments, the control unit 246 includes a user interface (not shown). In embodiments, the control unit 246 includes a printed circuit board (PCB) (not shown). The PCB may include a processor unit (e.g., 310 shown in FIG. 3). In embodiments, a pressure transducer (e.g., 340 shown in FIG. 3) is electrically coupled to the PCB.

Referring to FIG. 3, an embodiment of the canister assembly 240 illustrated in FIG. 2 is shown and includes a control unit 246 and a collection canister 242. Canister assembly 240 may be coupled via an extension assembly 230 to a dressing assembly (e.g., 12 shown in FIG. 1) to apply negative pressure to a wound to facilitate healing of the wound in accordance with various treatment modalities.

Collection canister 242 includes a chamber 335 (also referred to herein as first chamber 335) to collect wound fluids from the dressing assembly. A chamber top 336 may be disposed over the chamber 335. In embodiments, the collection canister 242 also includes a second chamber 325. Second chamber 325 may be defined at least in part by the chamber top 336 of the first chamber 335 and a bottom wall 326 of the control unit 246.

Control unit 246 includes a suction pump 360, a pump inlet conduit 372, a pump outlet conduit 362, a first filter element 376, a pressure sensor 340, a pressure sensor conduit 352 and a second filter element 354. Control unit 246 may also include a user interface (not shown). Pump inlet conduit 372 provides fluid communication between the suction pump 360 and the first filter element 376. Exhaust air from the pump 360 is vented through an exhaust port (not shown) via the pump outlet conduit 362. Pump outlet conduit 362 may be coupled to one or more filters (not shown) for filtering the exhaust air from the pump 360. Pressure sensor conduit 352 provides fluid communication between the pressure sensor 340 and the second filter element 354. Any suitable device capable of detecting pressure may be utilized for the pressure sensor 340, including, but not limited to, a pressure switch, transducer or transmitter.

The first filter element 376 may be disposed in the second chamber 325 of the collection canister 242. Additionally, or alternatively, the second filter element 354 may be disposed in the second chamber 325 of the collection canister 242.

Pressure sensor 340 (also referred to herein as transducer 340) is in fluid communication with canister 242 to detect the vacuum level at the collection canister 242. In various embodiments, the transducer 340 is capable of measuring vacuum levels within a chamber 335 of the collection canister 242 independent of the orientation of the canister assembly 240. In embodiments, the transducer 340 generates an electrical signal that varies as a function of vacuum level at the collection canister 242, which is communicated to the processor unit 310 of the control unit 246. Logic associated with the transducer 340 and the pump 360 may reduce the speed of the pump 360 or stop operation of the pump 360 in response to the vacuum level detected by the transducer 340.

Collection canister 242 includes a suction port 374 to communicate with the chamber 335 and the suction pump 360, a first valve 382, a canister inlet conduit 338, a canister inlet port 334 coupled to the inlet conduit 338 to introduce the wound fluids from the dressing assembly into the chamber 335, a second valve 384, and a pressure sensor port 396 to communicate with the chamber 335 and the transducer 340. In one embodiment, the first and second valves 382, 384 are mechanical valves, such as, for example, one-way flap valves. Pressure sensor port 396 is positioned at a “T”-off point 392 between the canister inlet conduit 338 and the canister inlet port 334. When the control unit 246 and the collection canister 242 are operablely coupled to each other, the transducer 340 is in fluid communication with the “T”-off point 392, as is described in more detail below.

Canister assembly 240 also includes a first connecting channel 378 and a second connecting channel 356. First connecting channel 378 is adapted to provide fluid communication between the first filter element 376 and the first chamber 335 of the collection canister 242, when the control unit 246 and the collection canister 242 are operablely coupled to each other. Second connecting channel 356 is adapted to provide fluid communication between the second filter element 354 and the pressure sensor port 396, when the control unit 246 and the collection canister 242 are operablely coupled to each other. In one embodiment, the first connecting channel 378 includes a plunger member “P1” positioned in an end portion thereof and adapted to engage the first valve 382 when the control unit 246 and the collection canister 242 are joined together. For example, plunger member “P1” may be sized and shaped to open the first valve 382, thereby enabling fluid communication between the first filter element 376 and the collection canister 242. In one embodiment, the plunger member “P1” includes an elongated conically-tapered distal end of sufficient length to enter the body of the first valve 382, when the control unit 246 and the collection canister 242 are joined together.

In one embodiment, the second connecting channel 356 includes a plunger member “P2” positioned in an end portion thereof and adapted to engage the second valve 384 when the control unit 246 and the collection canister 242 are joined together. For example, plunger “P2” may be sized and shaped to open the second valve 384, thereby enabling fluid communication between the second filter element 354 and the pressure sensor port 396. In one embodiment, the plunger member “P2” includes an elongated conically-tapered distal end of sufficient length to enter the body of second valve 384, when the control unit 246 and the collection canister 242 are joined together.

Canister assembly 240 may be constructed from a variety of materials, such as, for example, Lucite™ polycarbonate, metals, metal alloys, plastics, or other durable materials capable of withstanding forces applied during normal use, and may have some capability of withstanding possibly excessive forces resulting from misuse.

Control unit 246 includes a processor unit 310. In embodiments, the processor unit 310 is electrically coupled via a transmission line 341 to the transducer 340 and electrically coupled via a transmission line 361 to the suction pump 360. Processor unit 310 may include any type of computing device, computational circuit, or any type of processor or processing circuit capable of executing a series of instructions that are stored in a memory (not shown) of the control unit 246. The series of instructions may be transmitted via propagated signals for execution by one or more processors for performing the functions described herein and to achieve a technical effect in accordance with the present disclosure. In an embodiment of the canister assembly 240 depicted in FIG. 3, no electrical signals are transmitted between the control unit 246 and the canister 242. Collection canister 242 may be disposable. In an alternate embodiment (not shown), the transducer 340 is disposed within the second chamber 325 of the collection canister 242, and electrical signals indicative of the negative pressure being measured are transmitted from said transducer located within the second chamber 325 of the collection canister 242 to the control unit 246.

Control unit 246 is capable of controlling the vacuum level in the collection canister 242 independent of the orientation of the canister assembly 240. In an embodiment of the canister assembly 240 depicted in FIG. 3, the transducer 340 is in fluid communication with the “T”-off point 392 between the canister inlet conduit 338 and the canister inlet port 334. When the collection canister 242 contains exudate and the canister assembly 240 is tilted, e.g., relative to an upright position of the collection canister 242, there is a possibility that exudate may backflow into the canister inlet port 334. The likelihood of an occurrence of exudate backflow, which may occlude or partially occlude the canister inlet port 334, may depend on various factors such as the volume of exudate in the collection canister 242, characteristics of the exudate, and the angle of orientation of the canister assembly 240. During operation of the canister assembly 240, exudate backflow that occludes or partially occludes the canister inlet port 334 may be prevented from reaching the “T”-off point 392, e.g., the force of air flow via the canister inlet conduit 338 is in the direction of the canister inlet port 334, which would tend to prevent the exudate backflow from reaching the “T”-off point 392. Since the exudate does not occlude the transducer “T”-off point 392 in this situation, the transducer 340 can continue to monitor vacuum effectively since the transducer 340 may substantially continuously operate in fluid communication with the “T”-off point 392. In embodiments, the pressure sensor port 396, connecting channel 356, and pressure sensor conduit 352 all have air-tight connections, which may help to minimize the amount of exudate that can enter the pressure sensor port. Exudate will only enter the pressure sensor port 396 if it can displace the air already present, so exudate entry into pressure sensor port 396 would be minimal. This phenomena is not only true when the pump 360 is on, but is true also when the pump 360 is off while the control unit 246 and collection canister 242 are operably coupled to each other. The transducer 340 can continue to monitor vacuum effectively independent of orientation of canister assembly 240, since the transducer remains in fluid communication with the “T”-off point as described in this disclosure.

In embodiments, at least a portion of the inlet conduit 338 is disposed in the second chamber 325 of the collection canister 242. In embodiments, the “T”-off point between the inlet conduit 338 and the inlet port 334 is disposed in the second chamber 325 of the collection canister 242. In embodiments, the pressure sensor port 392 is disposed in the second chamber 325.

The control unit 246 includes a suction pump 360 to provide negative pressure. Suction pump 360 may provide negative pressure produced by a piston drawn through a cylinder. Suction pump 360 may be a manual pump or an automated pump. The automated pump may be in the form of a portable pump, e.g., a small or miniature pump that maintains or draws adequate and therapeutic vacuum levels. The pump may be a peristaltic pump or a diaphragm pump. In one embodiment, the suction pump 360 is a portable, lightweight, battery-operated, direct current (DC) motor-driven pump.

The user turns ON the canister assembly 240 by pressing a power button (not shown). When the power button is pressed, the control unit 246 performs a series of internal checks during power up. In one embodiment, after successfully completing the power-up tasks, the control unit 246 turns on the pump 360 using the stored settings. At initial activation of the canister assembly 240, the stored settings are the default settings. In one embodiment, the default settings for controlling the pump 360 are 80 mmHg and continuous mode. In one embodiment, the currently stored vacuum level setting can be altered by the user, e.g., to 50 mmHg. In one embodiment, the currently stored mode setting can be altered by the user, e.g., to an alternating mode.

In an embodiment shown in FIG. 3, the suction pump 360 is coupled to the first filter element 376, which is located within the control unit 246. In an alternate embodiment (not shown), the first filter element 376 is located within the collection canister 242, e.g., positioned in the second chamber 325. The first filter element 376 may include one or more filters and is configured to substantially prevent entry of exudate into the suction pump 360. A variety of filters can be used for the first filter element 376. In one embodiment, the first filter element 376 includes a hydrophobic filter that substantially prevents fluids from entering into the suction pump 360 and potentially causing damage to electronics or pneumatic components. In embodiments, the control unit 246 stops operation of the suction pump 360 when the first filter element 376 becomes occluded.

Transducer 340 is coupled to the second filter element 354, which is located within the control unit 246. In an alternate embodiment (not shown), the second filter element 354 is located within the collection canister 242, e.g., positioned in the second chamber 325. In one embodiment, the second filter element 354 is a hydrophobic filter that substantially prevents fluid contamination of the transducer 340. Transducer 340 monitors the vacuum level at the pressure sensor port 396. Pressure sensor port 396 is in fluid communication and close proximity to the canister inlet port 334 via the “T”-off point 392. The measured vacuum level at the pressure sensor port 396 may be substantially the same as the vacuum level within the collection canister 242.

Although embodiments of the present disclosure have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. A portable negative pressure wound therapy system, comprising:
a dressing assembly for positioning over a wound to apply a negative pressure to the wound; and
a canister assembly including:
a control unit;
a vacuum source disposed in the control unit;
a pressure sensor in communication with a processor unit of the control unit;
a collection canister having an inlet conduit in fluid communication with the dressing assembly, a first chamber to collect wound fluids from the dressing assembly, an inlet port coupled to the inlet conduit to introduce the wound fluids from the dressing assembly into the first chamber, a suction port to communicate with the first chamber and the vacuum source; and
a pressure sensor port to communicate with the first chamber and the pressure sensor, the pressure sensor port in fluid communication with a “T”-off point between the inlet conduit and the inlet port.

2. The portable negative pressure wound therapy system of claim 1, wherein the control unit monitors and controls a negative pressure within the first chamber of the collection canister.

3. The portable negative pressure wound therapy system of claim 1, wherein the canister assembly further includes:
a first filter element to prevent wound fluids from entering into the vacuum source; and
a first connecting channel to provide fluid communication between the first filter element and the first chamber of the collection canister, when the control unit and the collection canister are operablely coupled to each other.

4. The portable negative pressure wound therapy system of claim 3, wherein the canister assembly further includes:
a first valve, the first valve associated with the suction port,
wherein the first connecting channel includes a first plunger member positioned in an end portion thereof, the first plunger member to engage the first valve when the control unit and the collection canister are joined together.

5. The portable negative pressure wound therapy system of claim 4, wherein the canister assembly further includes:
a second filter element to substantially prevent fluid contamination of the pressure sensor; and
a second connecting channel to provide fluid communication between the second filter element and the pressure sensor port, when the control unit and the collection canister are operablely coupled to each other.

6. The portable negative pressure wound therapy system of claim 5, wherein the first and second filter elements are disposed in the control unit.

7. The portable negative pressure wound therapy system of claim 5, wherein the canister assembly further includes:
a top wall disposed over the first chamber; and
a second chamber defined at least in part by the top wall of the first chamber and a bottom wall of the control unit.

8. The portable negative pressure wound therapy system of claim 7, wherein the first filter element is disposed in the second chamber.

9. The portable negative pressure wound therapy system of claim 7, wherein the second filter element is disposed in the second chamber.

10. The portable negative pressure wound therapy system of claim 7, wherein the top wall of the first chamber includes the suction port.

11. The portable negative pressure wound therapy system of claim 7, wherein the top wall of the first chamber includes the inlet port.

12. The portable negative pressure wound therapy system of claim 11, wherein at least a portion of the inlet conduit is disposed in the second chamber.

13. The portable negative pressure wound therapy system of claim 12, wherein the “T”-off point between the inlet conduit and the inlet port is disposed in the second chamber.

14. The portable negative pressure wound therapy system of claim 7, wherein the pressure sensor port is disposed in the second chamber.

15. The portable negative pressure wound therapy system of claim 5, wherein the canister assembly further includes:
a second valve, the second valve associated with the pressure sensor port,
wherein the second connecting channel includes a second plunger member positioned in an end portion thereof, the second plunger member to engage the second valve when the control unit and the collection canister are joined together.

16. The portable negative pressure wound therapy system of claim 15, wherein the first and second valves are mechanical valves.

17. The portable negative pressure wound therapy system of claim 1, wherein the pressure sensor is a pressure transducer.
